(19) United States
(12) Patent Application Publication
(10) Pub. No.: US 2022/0177818 A1
(43) Pub. Date: Jun. 9, 2022

(54) OPERATION PROCESS FOR A CELL CULTIVATION SYSTEM

(71) Applicant: THE AUTOMATION PARTNERSHIP (CAMBRIDGE ) LTD., Hertfordshire (GB)

(72) Inventors: Jens-Christoph Matuszczyk, Göttingen (DE); Gerhard Greller, Göttingen (DE)

(21) Appl. No.: 17/600,480

(22) PCT Filed: Mar. 31, 2020
(86) PCT No.: PCT/EP2020/059149

§ 371 Date: Sep. 30, 2021

(30) Foreign Application Priority Data
Apr. 1, 2019 (EP) 19166531.4

(51) Int. Cl. C12M 1/32; C07K 1/36; C07K 1/16; C12M 1/36

(52) U.S. Cl. CPC C12M 23/12; C12M 41/48; C07K 1/16; C07K 1/36

(57) ABSTRACT

The present invention relates to an operation process for a cell cultivation system, the cultivation system comprising two or more cultivation vessels for the production of at least one biologic agent and/or cell, which cultivation vessels comprise cells in a suitable cultivation medium, the process comprising the steps of taking two or more liquid samples from two or more or cultivation vessels, optionally, purifying the liquid samples, analyzing at least one sample to acquire data relating to at least one system parameter indicative for at least one of nutrient status and/or medium quality of the cultivation medium, or cell density, or cell viability and/or one product parameter indicative for biologic agent quality and/or cell quality, and, adjusting, preferably in real-time, at least one process parameter and/or at least one feeding input in at least one cultivation vessel of the cultivation system, or of a subsequent cultivation system.

### FIELD OF THE INVENTION

The present application relates to the field of cell cultivation systems suitable for culturing cells,

### BACKGROUND

Generally, the optimization of a bioprocess, e.g., the operation process for a cell cultivation system, is subject to a large number of variables and adjustable process parameters, all of which have the potential to affect the process quality, including yield, cell viability and product quality.

In particular, Product quality is an important parameter, when it comes to the production of biosimilars, i.e., large protein molecules which are generic versions of respective branded molecules. In differences to true generics, i.e., generic versions of small molecular drugs, which are relatively easy to manufactures and are generally considered to be true copies of the respective branded drug, biosimilars are often considered to be only similar, although on a very high level, to their respective branded molecule.

This is because, even if the amino acid sequence, or the sequence of the encoding cDNA, is identical between the biosimilar and the branded molecule, the production process affects the molecule—sometimes abbreviated in the slogan “the product is the process”.

Generally, even with conserved amino acid sequence or the sequence of the encoding cDNA, the resulting products can differ from one another in parameters as their glycosylation pattern, disulfide bonds, C- or N-terminal heterogeneity, to name a few. These variabilities depend not only on the cell type used for expression, but also on the process parameters, culture media and so forth.

It is hence one object of the present invention to provide tools and methods to better understand the effect of process parameters, culture media and so forth, on process quality, including yield, cell viability and product quality, of a cell culture process.

It is one further object of the present invention to provide tools and methods to develop cell culture processes which allow the manufacture of biosimilars that provide a sufficient degree of similarity with a given branded molecule.

It is one further object of the present invention to provide tools and methods which allow a better understanding of the different factors in cell culture that have an influence on process quality, including yield, cell viability and product quality, of a cell culture process.

These and further objects are met with methods and means according to the independent claims of the present invention. The dependent claims are related to specific embodiments.

### SUMMARY OF THE INVENTION

The present invention provides an operation process for a cell cultivation system. Features, advantages and benefits of such process are described herein,

### BRIEF DESCRIPTION OF THE FIGURES

FIG. 1 shows an operation process for a cell cultivation system. The cell cultivation system comprising two or more cultivation vessels for the production of at least one biologic agent, which cultivation vessels comprise cells in a suitable cultivation medium,

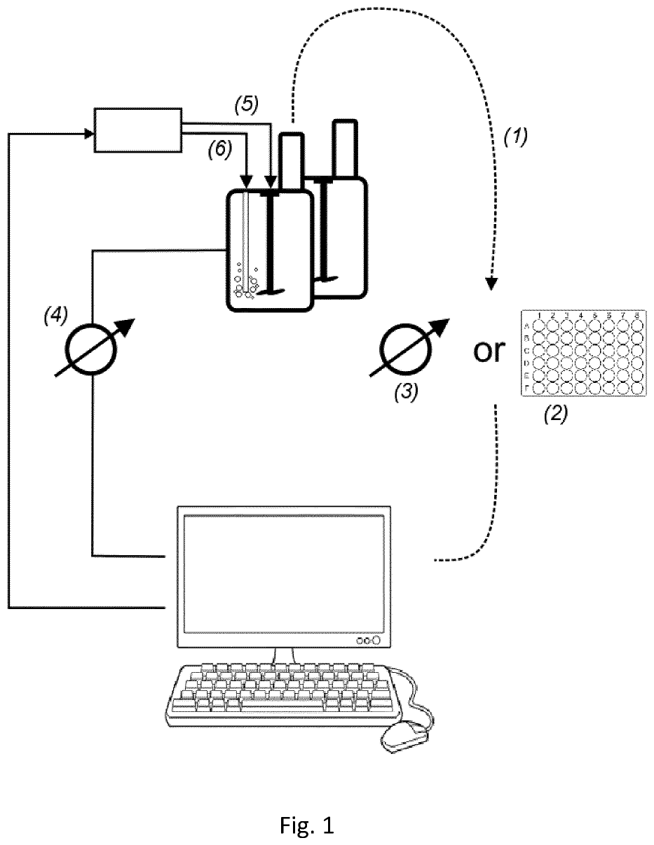

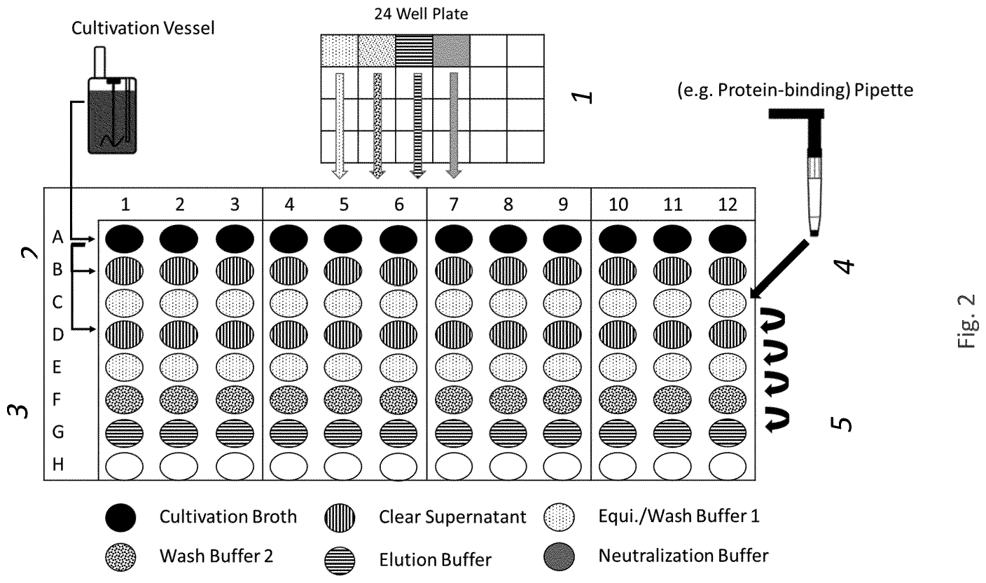

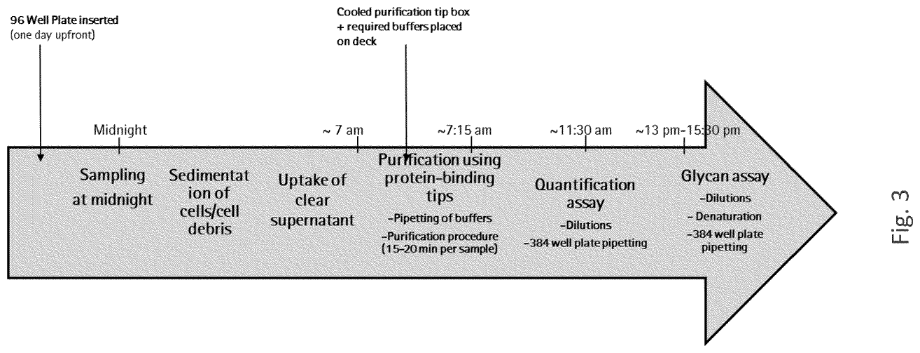

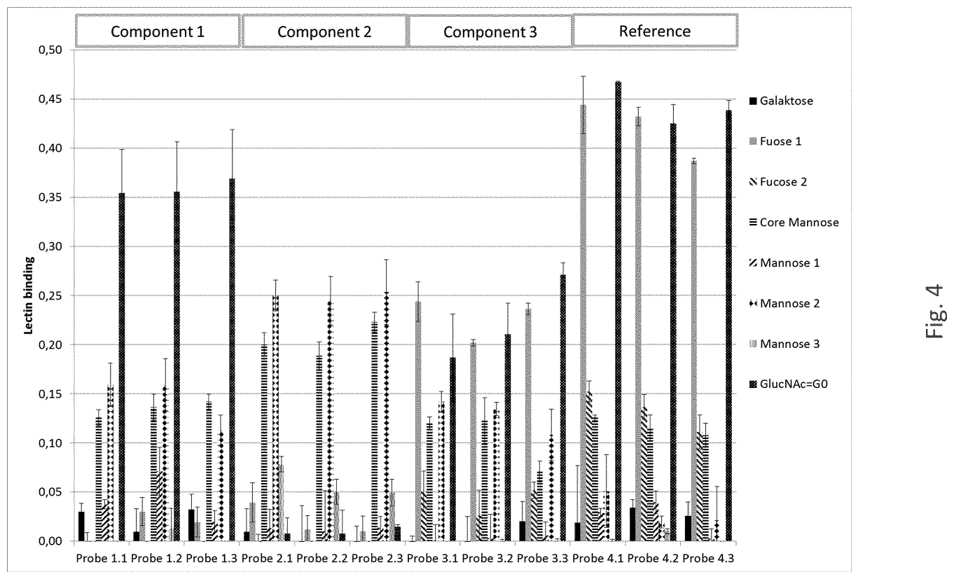

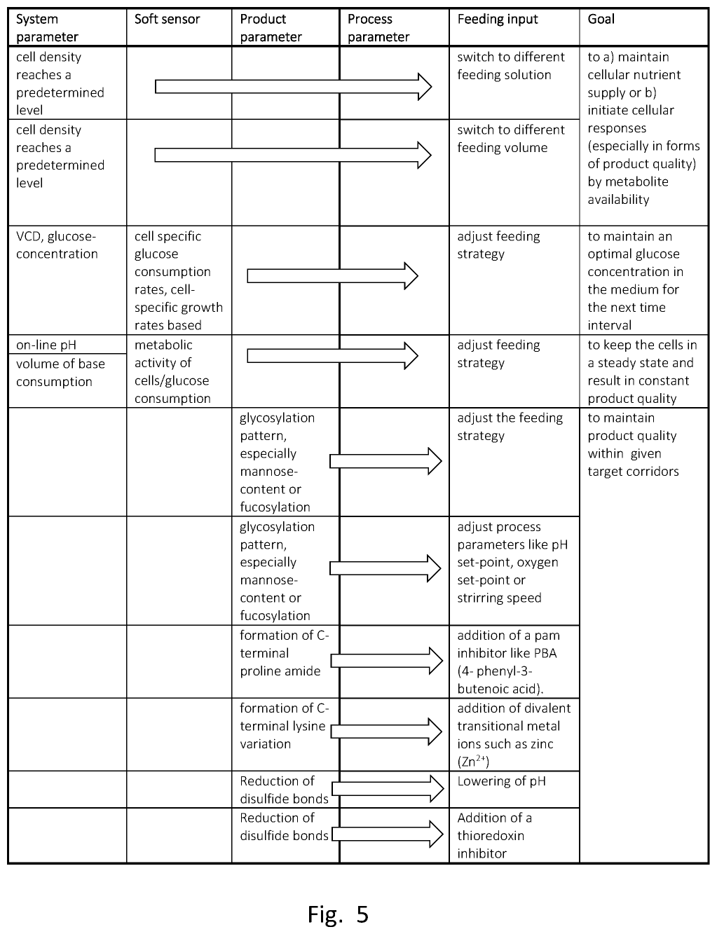

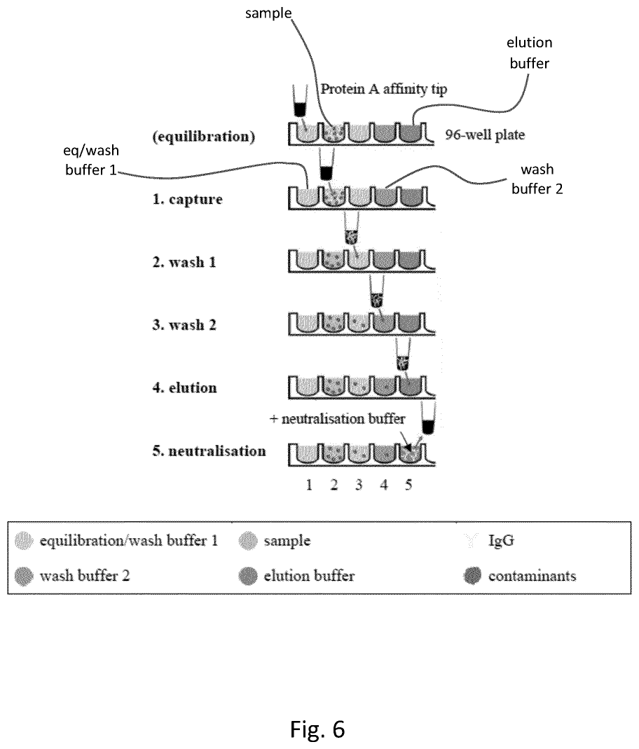

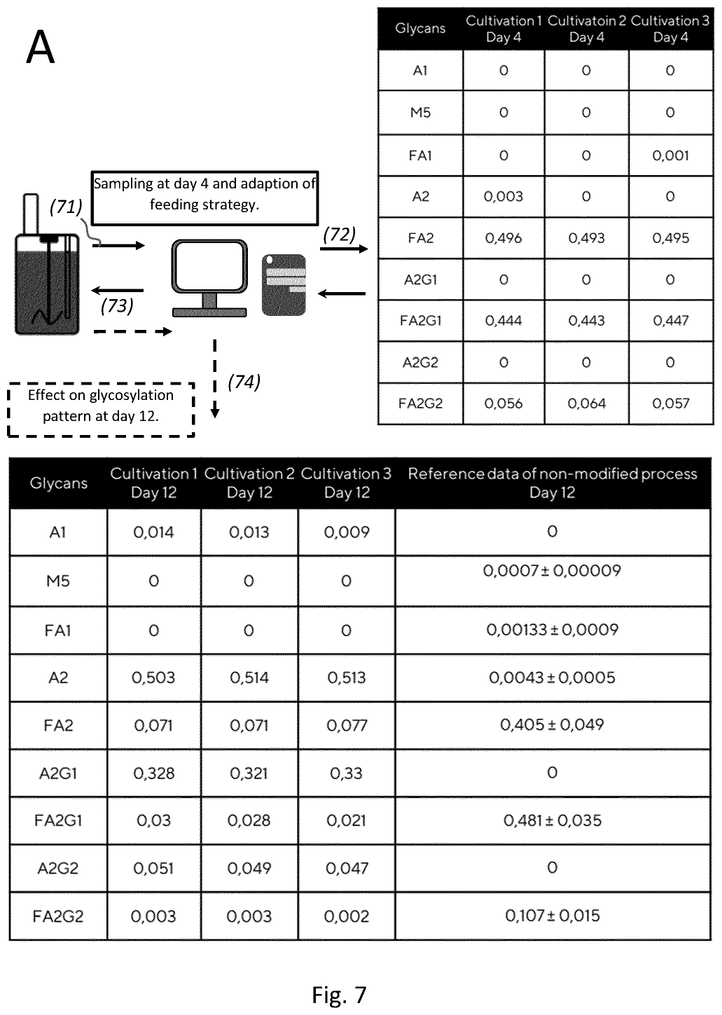

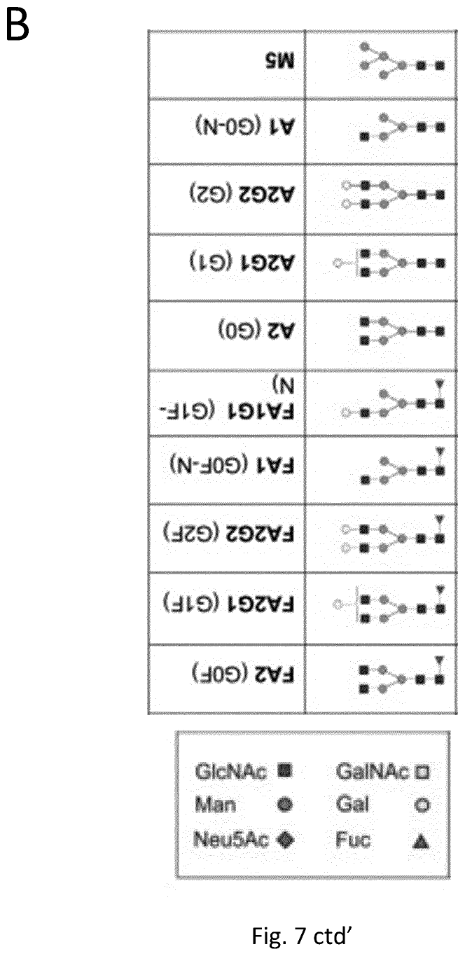

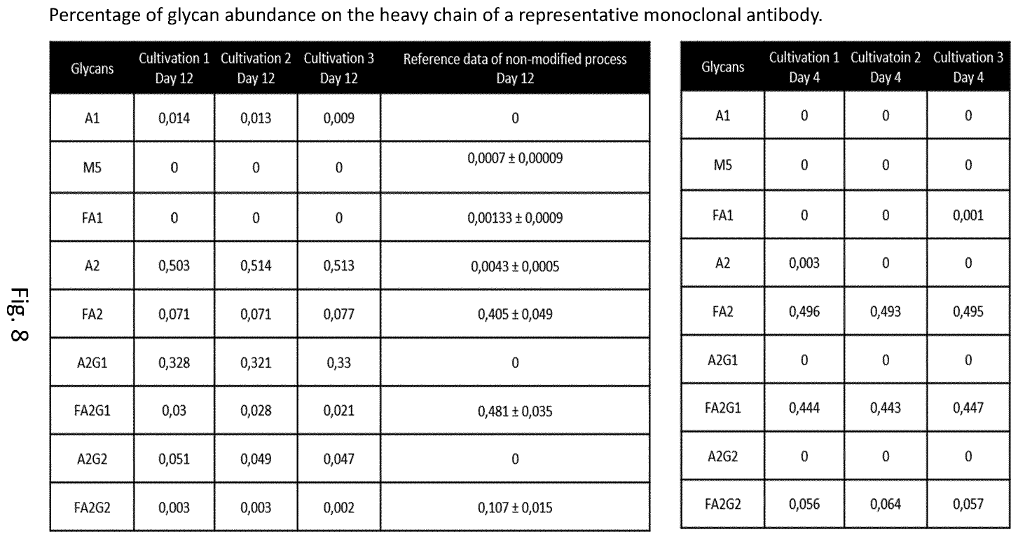

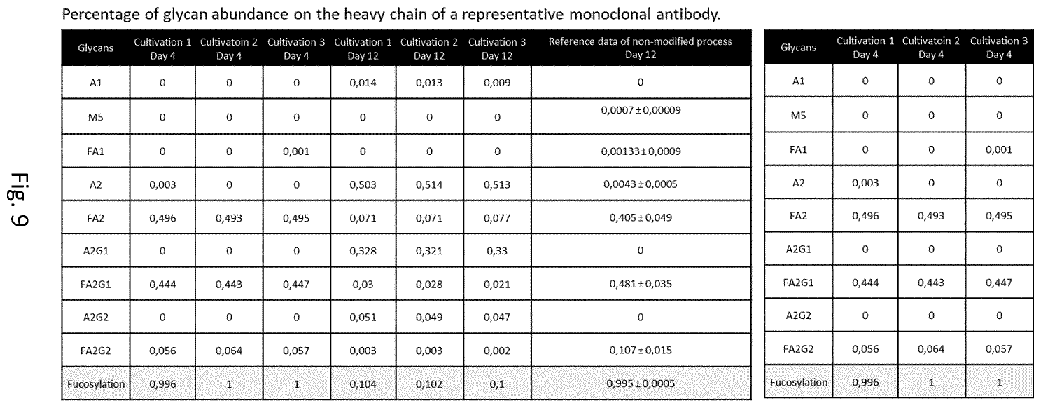

The process comprises the steps of taking two or more liquid samples from two or more or cultivation vessels (1); and analyzing at least one sample to acquire data relating to at least one system parameter (3) indicative for at least one of nutrient status and/or medium quality of the cultivation medium, or cell density, or cell viability and/or one product parameter (2) indicative for biologic agent quality.

Furthermore, optionally, at least one further system parameter (4) is measured on-line in at least one of the cultivation vessels.

In response to the outcome of that step, at least one process parameter (5) and/or at least one feeding input (6) in at least one vessel of the cell cultivation system is adjusted.

FIG. 2 shows an exemplary procedure for the purification of a biological agent, namely a monoclonal antibody from a cell culture, in microtiter plates. In a first step, liquid samples are transferred from the cell cultivation system to an array of reaction vessels, i.e., a microtiter plate, by means of a robotic liquid handler. In row A of reaction vessels, sedimentation of cells and debris takes place, and the clear supernatant is then transferred to a rows B and D of reaction vessels.

A pipetting robot using Protein A coated pipette tips (e.g., Aspire Protein A Tips by ThermoFisher) is then used to soak in supernatant comprising the antibody from row D. Antibodies bind to the Protein A coating, and after soaking and washing in rows E and F, the purified antibody is eluted into the reaction vessel in row G.

From there, samples are taken to acquire data related to a product parameter indicative for biologic agent quality, e.g., by MS (Mass Spectrometry), HPLC (High Performance Liquid Chromatography) or a Glycan Assay.

FIG. 3 shows an exemplary timeline of the automated sampling of liquid samples and subsequent at-line analysis to acquire data relating to inter alia product parameters indicative for biologic agent quality (here: Glycosylation pattern). The whole at-line process is designed to last less than 15 hrs, but can last shorter dependent of the particular specifications and conditions. In response to the outcome of the analysis step, at least one process parameter and/or at least one feeding input in at least one vessel of the cell cultivation system is then adapted, to maintain or change the respective product parameter.

FIG. 4 shows the glycan structure of various process samples after automated purification using Protein A pipette tips in a liquid handler. For analysis, a lectin-based glycan assay was used.

FIG. 5 shows some examples of system parameters, soft sensors and product parameters as acquired in the process according to the invention, and process parameters and feeding inputs as adjusted in the process according to the invention.

FIG. 6 shows a simplified scheme of the purification process steps performed on a micotiter plate. The buffers went through the resin of the pipette tip column consecutive back and forth. After elution, neutralisation buffer was added.

FIG. 7 shows results of A feedback experiment that was conducted to demonstrate the feasibility of the claimed method.

FIGS. 8 and 9 shows the respective data in more details.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to a first aspect of the invention, an operation process for a cell cultivation system is provided, the cell cultivation system comprising two or more cultivation vessels for the production of at least one biologic agent and/or cell, which cultivation vessels comprise cells in a suitable cultivation medium. The process comprises the steps of
a) taking two or more liquid samples from two or more or cultivation vessels
b) optionally, purifying the liquid samples
c) analyzing at least one sample to acquire data relating to at least
   one system parameter indicative for at least one of nutrient status and/or medium quality of the cultivation medium, or cell density, or cell viability and/or
   one product parameter indicative for biologic agent quality and/or cell quality, and
d) in response to the outcome of step c), adjusting, preferably in real-time, at least one process parameter and/or at least one feeding input in at least one vessel of the cell cultivation system, or of a subsequent cultivation system.

This process is essentially an “at-line” process, meaning that at least one analysis step is provided which entails diversion and collection of samples from the cultivation vessels. Collected portions are, for example, collected in sample vials for subsequent analysis. As discussed below, further on-line analysis steps can be used to complement the method.

As used herein, the term “system parameter” relates to a parameter that is indicative for at least one of nutrient status, medium quality of the cultivation medium, cell density, cell viability.

As used herein, the term “product parameter” relates to a parameter that is indicative for biologic agent quality.

As used herein, the term “process parameter” relates to a parameter that can be adjusted by an operator.

As used herein, the term “feeding input” relates to the input of a medium, preferably a fluidic one like a liquid or a gas.

As used herein, the term “in real time” is understood to mean that the process parameter or feeding input is adjusted within seconds, preferably within one second.

As used herein, the term “adjusting a parameter or feeding input in response to the outcome of the analysis step” can mean that either
a) a respective process parameter or feeding input is modified, to maintain or change the system parameter or product parameter, or
b) a respective process parameter or feeding input is maintained, to maintain or change the system parameter or product parameter.

In one embodiment, the cell cultivation system is a “bioreactor system” suitable for culturing cells. Generally, the term “cell cultivation system” encompasses all types of systems that are suitable for cultivating cells.

The term may encompass systems that are relatively complex, as they include for example stirrers or other equipment. These types of vessels will be called bioreactors, and the resulting vessel systems having two or more such bioreactors are called “bioreactor systems”.

The term may also encompass systems that are relatively simple, as they lack for example stirrers or other equipment.

According to an embodiment of the invention, in step a), the liquid samples are transferred to an array of reaction vessels for purification and/or analysis. For such purpose, a pipetting robot can for example be used, as will be discussed below.

According to an embodiment of the invention, in step a), the liquid samples are taken in a serial manner for purification and/or analysis, and are processed serially.

According to further embodiments of the invention, said array of reaction vessels comprises at least one of
a microtitre plate with two or more wells
a series of sample cups or sample vials, and/or
an array of microreaction tubes.

As used herein, the term “sample cups or sample” relates to small vessels that can be filled with sample liquid. As used herein, the term “array of microreaction tubes” relates to an array of sealable tubes or vials, like Eppendorf tubes or the like.

According to further embodiments of the invention, the biologic agent is selected from the group consisting of
a biomolecule (such as mAB, recombinant protein, proteins, polypeptides and nucleic acids agents)
a cell-based product (such as cells, comprising immune and stem cells, both as natural or transduced cells; as well as exosomes), and/or
a vaccine (such as a virus, a virion or a virus-like particle).

According to further embodiments of the invention, the process parameter that is adjusted in step d) is at least one selected from the group consisting of:
cultivation temperature set-point
stirring speed
stirrer blade tip speed
pH set-point
DO set-point
viable cell density set-point
conductivity
osmolality
specific power input
, and/or
bubble size.

According to further embodiments of the invention, the feeding input that is adjusted in step d) is at least one selected from the group consisting of:
O₂ gassing rate
compressed air gassing rate
CO₂ gassing rate
N₂ gassing rate
input of feed solution
composition of feed solution
addition or omission of individual ingredients in feed solution
addition of protective agents, e.g., blockpolymers
input of pH buffer,
addition of components related to increased productivity or prevention of cell death,
addition of proliferation stimulators,
addition of differentiation factors, and/or
addition of specific inhibitors.

Inhibitors can have different purposes. In general, they will be added to inhibit a particular reaction that may have a negative impact on product quality. In the following, a few examples will be discussed.

As discussed elsewhere, the formation of C-terminal proline amide residues is a major cause for heterogeneity in therapeutic antibodies. This phenomenon is discussed in WO2012062810A2, the content of which is incorporated herein by reference.

Also in said reference, inhibitors are discussed to avoid the formation of C-terminal proline amide residues. One class of these inhibitors are PAM inhibitors (peptidylglycine alpha amidating monooxigenase), like PBA (4-phenyl-3-butenoic acid).

As discussed elsewhere, the formation of C-terminal lysine variation is another major cause for heterogeneity in therapeutic antibodies. This phenomenon is discussed in Luo et al (2012), as well as in WO2012147053A1, the content of both of which is incorporated herein by reference. Also in the latter reference, inhibitors are discussed to avoid the formation of C-terminal lysine variation. Such inhibitors are e.g., divalent transitional metal ions such as zinc (Zn²⁺).

As discussed elsewhere, disulfide bonds recombinantly produced proteins are subject to reduction. This phenomenon is discussed in EP2188302B1, the content of which is incorporated herein by reference. Also in the latter reference, inhibitors are discussed to avoid the reduction of disulfide bonds, like thioredoxin inhibitors, including direct inhibitors of thioredoxin, specific inhibitors of thioredoxin reductase, or cupric sulfate.

This phenomenon is discussed in EP2586788B1, the content of which is incorporated herein by reference. Therein, it is discussed to lower the pH of a cell culture fluid to avoid disulfide bond reduction.

A component related to increased productivity is for example Na-Butyrate. This agent produces reversible changes in morphology, growth rate, and enzyme activities of several mammalian cell types in culture. This phenomenon is discussed in Prasad and Sinha (1976), the content of which is incorporated herein by reference.

A component related to the prevention of cell death is for example valproic acid, which induces dynamic modulation of histone H3 and α-tubulin acetylation. This phenomenon is discussed in Yagi et al (2010), the content of which is incorporated herein by reference.

Proliferation stimulators are for example amines like putrescine, cadaverine, spermine, spermidine and β-phenylethylamine, which have been shown in small concentrations to stimulate proliferation, as disclosed by Fusi et al (2008).

Further, they facilitate cell culture in a medium that is protein-free and does not comprise oligopeptides, as disclosed in EP1974014B1, the content of which is incorporated herein by reference.

Differentiation factors can be of different types.

Growth differentiation factors (GDFs) are a subfamily of proteins belonging to the transforming growth factor beta superfamily that have functions predominantly in development. Several members of this subfamily that can be used in the present context have been described and named GDF1 through GDF15.
GDF1 is expressed chiefly in the nervous system and functions in left-right patterning and mesoderm induction during embryonic development.
GDF2 (also known as BMP9) induces and maintains the response embryonic basal forebrain cholinergic neurons (BFCN) have to a neurotransmitter called acetylcholine, and regulates iron metabolism by increasing levels of a protein called hepcidin.
GDF3 is also known as “Vg-related gene 2” (Vgr-2). Expression of GDF3 occurs in ossifying bone during embryonic development and in the thymus, spleen, bone marrow brain, and adipose tissue of adults. It has a dual nature of function; it both inhibits and induces early stages of development in embryos.
GDF5 is expressed in the developing central nervous system, with roles in the development of joints and the skeleton, and increasing the survival of neurones that respond to a neurotransmitter called dopamine.
GDF6 interacts with bone morphogenetic proteins to regulate ectoderm patterning, and controls eye development.
GDF8 is now officially known as myostatin and controls the growth of muscle tissue.
GDF9, like GDF3, lacks one cysteine relative to other members of the TGF-β superfamily. Its gene expression is limited to the ovaries, and it has a role in ovulation.
GDF10 is closely related to BMP3 and has a roles in head formation and, it is presumed, in skeletal morphogenesis. It is also known as BMP-3b.
GDF11 controls anterior-posterior patterning by regulating the expression of Hox genes, and regulates the number of olfactory receptor neurons occurring in the olfactory epithelium, and numbers of retinal ganglionic cells developing in the retina.
GDF15 (also known as TGF-PL, MIC-1, PDF, PLAB, and PTGFB) has a role in regulating inflammatory and apoptotic pathways during tissue injury and certain disease processes

Other differentiation factors that can be used in the present context include
T-cell differentiation factor CBF-β
Neu differentiation factor (NDF)
Erythroleukemic Cell Differentiation Factor Edf

According to an embodiment of the invention, at least one further system parameter is measured on-line in at least one of the cultivation vessels.

As used herein, the term “on-line” herein refers to an analysis step which does not require the taking of a sample from the cultivation vessel, but can be performed directly in or at the cultivation vessel.

According to further embodiments of the invention, said further system parameter measured on-line is at least one selected from the group consisting of
dissolved oxygen,
pH,
trypan blue staining
pCO₂
optical density
osmolality or osmolarity
cell permittivity or radio-frequency (RF) impedance
cultivation time
exhaust gas composition
O₂ consumption in the medium
culture medium temperature
stirrer speed, and/or
metabolite levels.

As used herein, permittivity is the measure of capacitance that is encountered when forming an electric field in a particular medium. In cell culture applications, an alternating electric field is applied to the culture which measures the resulting polarization and depolarization of cells and microorganisms through a permittivity reading (capacitance per area). This signal can be correlated to the viable cell density (VCD), because only viable cells can be polarized. Dead cells have a leaky membrane and cannot be polarized. This method is therefore insensitive to dead cells, cell debris, and microcarriers. Radio-frequency (RF) impedance is a related method. Such methods are disclosed, inter alfa, in Zeiser et al (1999), or Harris et al (1987), the contents of which are incorporated herein by reference. Devices suitable for that purpose are e.g. produced by Hamilton (Incyte permittivity sensor).

As used herein, optical density is the measure for the turbidity of a liquid medium. In cell culture applications, optical density is measured in a cell suspension at near infrared (NIR) wavelengths (i.e. >600 nm). All particles and molecules that scatter the NIR light are detected and can be correlated to the total cell density (TCD). Devices suitable for that purpose are e.g. produced by Hamilton (Dencytee sensor).

pH and dissolved oxygen (DO) can be measured with so-called sensor spots. pH and DO sensor spots are for example comprised in the ambr15 bioreactors supplied by Sartorius. They are mounted inside the vessels and measurements are taken through the transparent vessel wall. Therefore, optical sensors are minimizing the number of parts that need to be discarded. They usually consist of a thin layer containing an analyte-sensitive (oxygen, pH, or CO₂) dye. These sensor spots, which are excited by light of a certain wavelength, then fluoresce. If the sensor spot encounters a molecule of the respective analyte, the excess energy is transferred in a nonradiative way, decreasing or quenching the fluorescence signal. The respective reading systems comprise integrated optical modules applying LEDs, photodiodes, and polymer optical fiber to transfer light to the sensors and read the luminescence response. Such sensor spots are for example manufactured by PreSens Precision Sensing GmbH.

Exhaust gas can be determined from via a specific port by, e.g., mass spectrometry, gas chromatography or typical exhaust gas analysis Winckler et al (2013), the content of which is incorporated herein by reference This technique provides the ability to determine the oxygen uptake rate (OUR) and the carbondioxide evolution rate (CER) without interrupting the ongoing process.

According to one or more embodiments, the method further comprises, after step c) and before step d), the determination of a soft sensor parameter based on at least one system and/or product parameter, which soft sensor is used as a basis for the adaptation in step d).

The term “soft sensor” as used herein, often also called “virtual sensor”, describes a software or algorithm in which where several measurements are processed together, to obtain an integrated or processed output value that is then evaluated similar to a hardware sensor output. In cell culture applications, soft sensors are being used for a different purposes, as e.g., discussed in Ohadi et al (2015), Ohadi et al (2014), Gustaysson R (2018), Abu-Absi at al (2011), Kroll et al (2017), or Luttmann et al (2012), the content of each of which is incorporated by reference herein. Some typical such soft sensors are shown in the following table:

| Process or product parameters | Soft sensor |
|---|---|
| cell density and Glucose | cell-specific glucose consumption; |
| concentration | glucose specific lactate yield: |
| cell density and Lactate | cell-specific lactate production; |
| concentration | glucose specific lactate yield: |
| cell density | batch age; specifc growth rate |
| cultivation time | Extrapolated cell density |
| O₂ cascade stage and dissolved | Extrapolated cell density |
| oxygen | |
| cell density and substrate | cell-specific substrate consumption |
| concentration | |
| Viable cell density, dO and | cellular oxygen demand |
| gassing strategy | |

According to further embodiments of the invention, the liquid samples are taken with at least one of
a robotic liquid handler, and/or
a multi valve sample system.

As used herein, the term “robotic liquid handler” relates to a pipetting robot that can perform pipetting tasks in parallel or serial. Such robots are for example provided by Beckman Coulter (Biomek NXp Robotic Liquid Handler), Brand (Liquid Handling Station) and others.

As used herein, the term “multi valve sample system” relates to systems in which samples are taken from the cultivation vessels by means of fixed valve/tube connections, i.e., without a pipetting robot. Such systems are for example provided by Eppendorf (DASGIP).

According to an embodiment of the invention, the taking of two or more liquid samples is triggered by a given system parameter. Preferably, such system parameter is a system parameter that is measured on-line.

According to one other embodiment, the taking of two or more liquid samples is triggered by a timer.

According to an embodiment of the invention, at least one liquid sample comprises supernatant from one of the cultivation vessels. Such supernatant, ideally, comprises molecules that have been secreted by the cells into the medium, but does not comprise cells or debris.

According to further embodiments of the invention, the system parameter indicative for cell density is at least one selected from the group consisting of:
optical density (Lambert beer)
cell permittivity or radio-frequency (RF) impedance
transmission spectrum.
light scattering, and/or
trypan blue staining
   of e.g. a cell suspension. According to further embodiments of the invention, the system parameter indicative for cell viability is at least one selected from the group consisting of:
Trypan blue exclusion
cell permittivity or radio-frequency (RF) impedance and/or
transmission spectrum.

Trypan blue exclusion is for exampled disclosed in Strober (2001), the content of which is incorporated herein by reference.

According to further embodiments of the invention, the system parameter indicative for nutrient status and/or medium quality of the cultivation medium is at least one selected from the group consisting of
Glucose concentration
Concentration of at least one trace element, vitamin, organic acid or amino acid
Osmolality or osmolarity
Lactate concentration
Acetate concentration,
Ammonia concentration, and/or
pH value

Methods for the determination of these system parameters in cell culture applications are disclosed, inter alia, in Peuker et al. (2014), Konakosky et al (2016), as well as McGillicuddy et al (2017), the content of each of which is incorporated by reference herein.

As used herein, the term “trace element” relates to an element that is present in only a trace concentration. A trace concentration may be less than a level ordinarily or easily measured, for example the trace level may be <10⁻⁵, <10⁻⁶, <10⁻⁷ or <10⁻⁸M. The trace elements of the present invention are preferably present as ions or chelated complexes. The ions may be simple ions comprising only a single element or may be complex ions comprising two or more elements. Preferably the elements are transition metal elements, e.g., elements selected from the group consisting of Sc, Ti, V, Cr, Mn, Fe, Co, Ni, Cu₅ Zn, Ga, As, Se, Br, Al, Si, P, Y, Zr, Nb, Mo, Tc, Ru, Rh, Rb, Ce, Ag, Pd, Ag, Cd, In, Sn, Sb, F, Te, Au, Pt₅ Bi, Ir, Os, Re, W, Ta and Hf. Some elements may be present at more than trace amounts, i.e., >10⁻⁵ in a Ix concentration, in which case that element, e.g., Fe or Zn would not be considered a trace element, but may nonetheless be advantageously used with or as part of the present invention and thus may be included specifically in some aspects of the invention.

According to further embodiments of the invention, the biomolecule is a recombinant biomolecule, preferably selected from the group consisting of
an antibody, or fragment or derivative thereof, or an antibody mimetic, and/or
a growth factor, hormone or cytokine.

According to further embodiments of the invention, wherein the product parameter indicative for quality of the biomolecule is at least one selected from the group consisting of
Glycosylation pattern,
Aggregation
Deamidation
Oxidation
Fragmentation
Glycation
Charge Profile
Non-glycosylated heavy chain (NGHC)
Endotoxin concentration
Host cell protein concentration
Potency or antigen binding
formation of C-terminal proline amide residues
C-terminal lysine variation, and/or
Disulfide bond reduction.

As used herein, potency or antigen binding can be determined with methods known in the art., e.g., by Antigen binding assays, Fc functional testing; FcgR, C1q and FcRn binding, or by Cell-based potency assays.

As used herein, the glycosylation pattern can be determined with methods known in the art., e.g., lectin microarrays (Zhang et al 2016 (1)) and others (Zhang et al 2016 (2), Yang X et al 2016), the content of all of which is incorporated herein by reference. Other approaches encompass HPLC, LC-MS and LC-MS/MS, all of which are known to the skilled person.

The formation of C-terminal proline amide residues is a major cause for heterogeneity in therapeutic antibodies. This phenomenon is discussed in WO2012062810A2, the content of which is incorporated herein by reference.

C-terminal lysine variation is another major cause for heterogeneity in therapeutic antibodies. This phenomenon is discussed in Luo et al (2012), as well as in WO2012147053A1, the content of both of which is incorporated herein by reference.

The problem of disulfide bind reduction in recombinantly produced proteins is discussed in EP2188302B1, the content of which is incorporated herein by reference.

According to further embodiments of the invention, the cell-based product is selected from the group consisting of
a stem cell product
a CAR T cell product, and/or
an NK T cell product.

According to further embodiments of the invention, the product parameter indicative for quality of the cell-based product is at least one selected from the group consisting of
presence of one or more given surface markers, and/or
cell viability.

Such surface markers can for example be used to determine the status of the cells that are meant to be produced, e.g., as regards their potency state.

According to further embodiments of the invention, the product parameter indicative for quality of the vaccine is a Critical Quality Attribute (CQA).

As used herein, the term CQA relates to Critical Quality Attributes, which is a list of quality parameters that is checked in the process of vaccine quality management. Such parameters are e.g. published by the FDA (“Guidance for Industry, November 2009”), the content of which is incorporated herein by reference.

According to further embodiments of the invention, the cells in the cultivation vessels are at least one selected from the group consisting of
Bacterial cells
Yeast cells or filamentous funghi cells
Protozoan cells
Insect cells, and/or
Mammalian cells

According to further embodiments of the invention, the purification step comprises at least one step selected from the group consisting of
protein A based purification,
protein G based purification,
affinity tag based purification,
lectin based purification,
ion chromatography,
affinity chromatography, and/or
size exclusion chromatography

Protein A based purification methods have been developed to purify antibodies from cultivation media. The primary binding site for protein A is on the Fc region of antibodies, between the CH2 and CH3 domains. In addition, protein A binds human IgG molecules containing IgG F(ab′)2 fragments from the human VH3 gene family. Methods of using protein A for antibody purification are disclosed in Shukla et al (2007), the content of which is incorporated herein by reference.

Affinity tag based purification relates to tag-based methods including His tag and Strep tag. Such methods are e.g. disclosed in Kimple et al (2013), the content of which is incorporated herein by reference.

Lectin based purification is for example disclosed in Nascimento et al (2012), the content of which is incorporated herein by reference.

Ion chromatography, affinity chromatography and size exclusion chromatography are other methods to purify proteins from cultivation media. They are fully within the routine of the skilled person. An overview is provided in Coskun (2016), the content of which is incorporated herein by reference.

According to an embodiment of the invention, at least one of the cultivation vessels of the cell cultivation system is a single use bioreactor.

According to further embodiments of the invention, the cell cultivation system comprises at least 2, preferably 4 cultivation vessels. In one embodiment, the cell cultivation system comprises 12 cultivation vessels. In another embodiment, the cell cultivation system comprises 24 cultivation vessels.

According to further embodiments of the invention, cell cultivation system comprising two or more cultivation vessels is at least one selected from the group consisting of
ambr® 15 cell culture system
ambr® 250 cell culture system,
BioLector microbioreactor
DASGIP® parallel bioreactor
DASbox® Mini bioreactor system
Micro-24 microreactor, and/or
micro-Matrix.

The following table gives an overview of such cell cultivation systems:

| cell cultivation system | manufacturer |
|---|---|
| ambr ® 15 | Sartorius |
| ambr ® 250 | Sartorius |
| BioLector | m2p-labs |
| DASGIP ® | Eppendorf |
| DASbox ® | Eppendorf |
| Micro-24 | Pall Corporation |
| micro-Matrix | Applikon Biotechnology |

According to another aspect of the invention, a cell cultivation system suitable for operating the process according to the above description is provided.

This entails that such cell cultivation system provides technical features that make it suitable for operating said process. The technical features are described in detail herein.

According to one embodiment, the cell cultivation system is a bioreactor system suitable for culturing cells, the bioreactor system comprising two or more bioreactors.

According to another aspect of the invention, a method of manufacturing a biologic agent is provided, in which method a process or a cultivation system according to the above description is applied. This entails that the biological agent differs from other biological agents in a novelty conferring manner, because, as discussed above, the “product is the process”.

According to another aspect of the invention, a biologic agent made with the cell cultivation system according or the process according the above description is provided. Again, this entails that the biological agent differs from other biological agents in a novelty conferring manner, because, as discussed above, the “product is the process”.

### Experiments and Figures

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Any reference signs should not be construed as limiting the scope.

### CHO Cultivation

The seed and main cultivation procedures performed in this work were based on standardized processes by Sartorius. Besides these, the composition and preparation of the used media and solutions are stated.

### Seed and Main Culture

The used CHO DG44 cryos were delivered by Sartorius Stedim Cellca. The cells had been modified to synthesise and secrete an IgG1 type antibody. When delivered, the 1 mL cryo vials were in the cultivation passage eight and contained approximately 3×107 cells/mL. After thawing, the cell suspension was transferred into 10 mL prewarmed (36.8° C.) seed medium (SM). To remove the preservation medium, the suspension was then centrifuged for 3 minutes at 190×g. After decanting the supernatant, the cells were resuspended in 150 mL prewarmed SM (500 mL shake flask, Corning). The shake flask (SF) was then incubated (Certomat® CTplus, Sartorius) at the conditions stated in the following table

### Seed Culture Conditions for Incubation (Certomat® CTplus)

| parameter | setpoint |
|---|---|
| shaking rate | 120 rpm |
| temperature | 36.8° C. |
| humidity | 85% |
| pCO₂ | 7.5% |

Every three to four days, the seed culture was splitted and thereby transferred into the next passage. Hereby, a SF with prewarmed SM was inoculated with a certain volume of the passage before to start the new one with a VCD of around 2×105 cells/mL (Cedex HiRes Analyser, Roche). The cell splitting was repeated until the main culture was inoculated in passage thirteen.

For the performed main cultivations, different types of media were used in this work. Batch processes were performed in process medium (PM). The first three days of a fed-batch process were cultivated in only PM as well. After that, a daily addition of feed medium A (FMA) and feed medium B (FMB) was done. Also, a glucose solution was fed, once the corresponding level was below 6 g/L. When foam formation was detected, an antifoam solution was added.

### Solution and Media Composition

In the following, the compositions of all used media and solutions are stated according to the standard operation procedures of Sartorius. The exact formulas of these are not accessible for the user. The powder and other necessary chemicals were weighted (LA 5200P, Sartorius) and solved in reverse osmosis (RO) water from an in-house reverse osmosis system. Adding order and stirring times were fixed. In the preparation of all media, the adjustment of the pH values (pH-meter Basic PB-11, Sartorius) was done with a 2 M NaOH solution (Merck). The glucose level was controlled with the blood gas analyser ABL800 (Radiometer) and the osmolality with the Osmomat® 030 (Gonotec).

### Seed Medium

The first five steps of the cultivation cascade were done in SM. The components stated in table 3-2 were given to RO-water sequentially. After the adjustment of the pH value in a range of 6.9 to 7.3, the medium was stirred for 60 more minutes. The medium was then checked for an osmolality between 270 and 300 mOsmol/kg and a glucose concentration between 5.5 and 6.5 g/L. Afterwards, it was filtrated (Sartopore® 2 0.1 μm, Sartorius). For the cultivation passages eight to ten, 0.03 g/L methotrexate (MTX, Sigma-Aldrich) were added sterile for optimal maintenance of the selection pressure.

### Preparation of Seed Medium

| | | con- | stirring |
|---|---|---|---|
| | | centration | time |
| component | supplier | [g/L] | [min] |
| CHOKO Stock Culture Medium | SAFC | 20.040 | 60 |
| L-Glutamine | Sigma-Aldrich | 0.880 | 30 |
| NaOH Pellets | Merck | 0.240 | 30 |
| NaHCO₃ | Sigma-Aldrich | 1.800 | 15 |

### Process Medium

Table 3-3 states the components and their concentration solved in RO-water for the preparation of PM. The pH was adjusted to a value between 6.90 and 7.35. In a quality control before sterile filtration (Sartopore® 2 0.1 μm, Sartorius), the osmolality was controlled to be between 274 and 300 mOsmol/kg and the glucose concentration between 5.5 and 6.5 g/L.

### Preparation of Process Medium

| | | con- | stirring |
|---|---|---|---|
| | | centration | time |
| component | supplier | [g/L] | [min] |
| CHOKO Production Medium | SAFC | 22.310 | 60 |
| L-Glutamine | Sigma-Aldrich | 0.880 | 30 |
| 5M NaOH solution | Merck | 6.200 | 30 |
| NaHCO₃ | Sigma-Aldrich | 1.800 | 15 |

### Feed-Medium A

After solving the components stated in table 3-4 in RO-water, the pH was adjusted to a value between 6.5 and 6.8. In a quality control before sterile filtration (Sartopore® 2 0.1 μm, Sartorius), the osmolality was measured to range between 208 and 268 mOsmol/kg and the glucose concentration between 70 and 80 g/L. The density was set to 1.0558 g/mL.

### Feed Medium A

| | | concentration | stirring time |
|---|---|---|---|
| component | supplier | [g/L] | [min] |
| CHOKO FMA | SAFC | 154.120 | 60 |
| NaOH Pellets | Merck | 3.148 | 30 |

### Feed-Medium B

In table 3-5, the main components of FMB are presented. The pH value was adjusted between 11.0 and 11.4. The osmolality should be between 218 to 266 and the density 1.0494 g/mL.

### Feed Medium B

| | | concentration | stirring time |
|---|---|---|---|
| component | supplier | [g/L] | [min] |
| CHOKO FMB | SAFC | 94.600 | 60 |
| NaOH Pacts | Merck | 32.100 | 30 |

### Glucose Feed

For a 400 g/L concentrated glucose feed solution, 400 g of D-glucose (Sigma-Aldrich) were added to 772.0 mL of RO-water. Afterwards, it was sterile filtrated (Sartopore® 2 0.1 μL, Sartorius). The density of the solution was around 1.15 g/mL.

### Antifoam

2% Antifoam C solution was used against foam formation. For this, 66.7 mL of Antifoam C Emulsion (Sigma-Aldrich) was filled up with RO-water to the final volume of 1 L. Sterilisation was done by autoclaving at 121° C. for 20 minutes (Fedegari autoclave).

### Preliminary Implementation of Sub-Processes

Protein A affinity tips were attached to the ambr® 15 liquid handler for automated purification. However, some processing steps were required before the actual purification of the product in the cell culture supernatant, leading to the three main process steps seen in FIG. 3.1. After automated sampling of CHO cell culture broth performed by the liquid handler, the first step of the product processing was the separation of secreted IgG in the supernatant from cells and cell debris. This was intended to prevent clogging of the tip columns. An appropriate method for this clarification step was sedimentation because no user intervention was required. After sedimentation, the clear supernatant was taken up and purified by the liquid handler.

Following the HTS concept of the ambr® 15 system, all steps were performed on a 96-well microtiter plate located on the deck. A sterile 96-well polystyrene microplate with U-bottom and a mathematical well volume of 323 μL was chosen (Greiner Bio-One). The round-shaped well bottoms had no edges restraining liquid and were therefore appropriate for the purification processes. The clear plate also allowed the transparency and optimisation of the pipette tip depth for the uptake of the supernatant after sedimentation as well as for the purification process. The implementation methods for the sub-processes of sedimentation and purification are described in the following.

### Sedimentation

The necessary sedimentation time of the CHO cells was examined experimentally. Particular attention was paid to varying cell characteristics in different phases of growth as well as different cell concentrations. The experimental results were then adapted to mathematical approaches by introducing phase specific correction factors.

### Experimental Analysis

After sedimentation, the supernatant was further processed whereas the cells on the bottom of the well were discarded. To ensure a clear separation, the height offset of the pipette tip from the bottom had to be adjusted for the uptake of the supernatant. With the determined adjustment, actual tests on the sedimentation time were then performed.

### Application of the Purification Procedure

Protein A PhyTips® have been improved for specific instrument flow rates and handling. The application technology was adjusted to the ambr® 15 system, based on the product information. While the capture step should occur at a flow rate of 250 μL/min (˜4.17 μL/s) the suggested rate for the other steps is 500 μL/min (˜8.33 μL/s). In addition, the aspirate-and-dispense cycles vary by number. For the capture step, four cycles are recommended whereas for each wash step two and for the elution step five cycles are recommended.

All buffers necessary for the purification were supplied by PhyNexus together with the tips. The purification process was based on the five main steps discussed herein. Three different purification approaches were performed to apply the purification method. In all of them, sample (supernatant) saved from the shake flask cultivations of the sedimentation analysis was purified. With the first two approaches, the above stated recommended numbers of cycles were tested. For this, three trials were run with five cycles for each purification step and three were performed with the recommended numbers of cycles (four capture cycles, two times two wash cycles and five elution cycles). A process with five cycles was chosen in this evaluation as it was the highest number occurring in the recommendation. The numbers of cycles were set in the pipetting scheme specified in the experimental protocol. In the third approach, a preceding equilibration step was conducted with the intention to improve the effectivity of IgG binding. For this, wash buffer 1 was drawn up and spit at two cycles. When the tips were delivered from PhyNexus, the resins were stored in a drop of glycerol. In addition to the adjustment of the optimal pH value for binding, the equilibration step could therefore wash out or at least dilute the glycerol.

Except for the numbers of cycles and the additional equilibration step in the last approach, all trials were conducted with the same proceeding on a 96-well plate. First of all, 185 μL of buffers and supernatant were pipetted into the wells of the plate manually (Pipette Research® 1000 μL, Eppendorf). A phosphate wash buffer 1 of pH 7.4 was added into column 3 and for the approach with the equilibration step also in column 1. Sample, wash buffer 2 (saline solution) and elution buffer were given into the columns 2, 4 and 5. For the elution step, a phosphate buffer of pH 2.5 was applied. During the purification, the Protein A affinity tip serially moved to the well with the particular buffer and performed the defined aspirate-and-dispense cycles. Hence, the liquid handler moved from column 2 to 5 in case of no equilibration and from 1 to 5 with one. The pipetting volume was set to 300 μL to guarantee aspirating and dispensing of the complete liquid, despite the slightly different tip geometry and the retrofitting. After elution, 45 μL of a tris buffer solution that contained a pH value of 9.0 was added to neutralise the pH and keep the protein active.

Furthermore, the impact of remaining cells after clarification on the purification performance was examined. This should evaluate the need for the sedimentation step. For this, a 1 L SF (Corning) was inoculated with 0.3×10⁶ cells/mL of a seed culture in passage nine. The cultivation was performed similarly to the shake flask cultivations used for the sedimentation analysis. On the seventh cultivation day, cell suspensions with densities of approximately 1×105 cells/mL and 1×10⁴ cells/mL (counting chamber, Marienfeld superior) were prepared based on equation (3.1). With the same settings as in the third approach described above, three purification runs were then performed with samples containing 1×10⁴ cells/mL, three with 1×10⁵ cells/mL and three with clear supernatant.

### Performance Evaluation

For a better representation, triple determinations were made for each variant of the purification process. The IgG titers of the corresponding buffers and supernatants were measured via size exclusion chromatography (SEC) after the end of the process multiplying the applied volumes to the detected concentrations, the IgG amounts were then determined. Based on these, the recoveries were calculated with the following formula:

$r = \frac{{I}_{g} {G}_{e}}{{I}_{g} {G}_{s}} 100 \%$

| | | |
|---|---|---|
| r | recovery | [%] |
| IgGₑ | amount of purified IgG | [μg] |
| IgGₛ | amount of IgG in supernatant | [μg]. |

Next to the recovery, the performance of the purification via Protein A PhyTips® was evaluated by the purity. An estimation of purity was possible due to the comparison of the contaminants in the chromatograms, before and after purification. The method did not allow to draw conclusions in respect of the exact number of peaks in each chromatogram which represent contaminants, because molecule intensities could overlap. Furthermore, a quantification of the unknown substances was not possible as calibration series were not available. Taking both aspects into account, only an estimation of purity could be made. However, the comparison of the purification performance by the PhyTips® with an already established method helped to evaluate the purity. For this, small supernatant volumes were in addition purified with Protein A SpinTraps™ (GE Healthcare) by following the instruction manual. The pure protein solutions were analysed chromatographically as well. Chromatograms of average performances from the purification procedures via SpinTraps™ and PhyTips® were then compared.

### Fed-Batch Cultivations With Inhibitors of Glycosylation

The fed-batch was performed based on general guidelines from Sartorius. Ambr® 15 vessels containing 10 mL preheated PM were inoculated with seed culture suspension to a start VCD of approximately 0.3×106 cells/mL. From day three, the vessels were daily fed with 398 μL of FMA and 40 μL of FMB. When foam formation was detected, 20 μL of antifoam were added.

The amount of glucose solution that ensured a concentration of 6 g/L was fed once the level dropped below this limit. Furthermore, 480 μL volume was sampled on the days three to seven and ten for offline analysis. All regulating and pipetting steps were defined in an experimental protocol of the ambr® 15 software. The pH value was set to 7.1, the temperature to 36.8° C., the DO to 60% and the stirring to 1300 rpm. Furthermore, nitrogen and oxygen were aerated at rates of 0.15 mL/min and 75.0 mL/min.

In total, eleven vessels were cultivated. Two of them were cultivated with the original media and solutions. Moreover, experimental approaches with 2F-Peracetyl-Fucose, Kifunensine, and NGI-1 were performed as triple determinations. Each of the three modifiers was added to PM and FMA in the concentrations stated in table 3-6 and sterile filtrated (Minisart® 0.1 μm, Sartorius). Adding of the mentioned modifiers to FMB, additional glucose and antifoam was neglected because of the small volumes.

### Substances and Corresponding Concentrations used for the Modification of Glycosylation Patterns in the Fed-Batch Cultivation

| substance | supplier | concentration [μM] |
|---|---|---|
| 2F-Peracetyl-Fucose | Sigma-Aldrich | ~100 |
| Kifunensine | Sigma-Aldrich | ~20 |
| NGI-1 | Sigma-Aldrich | ~25 |

### Automated Sedimentation and Purification

For the processing, cell suspension of just one vessel per substance and one control was used because the technical process procedure was tested and not the biological reproducibility. The workflow of the clarification and the purification processes were integrated on a 96-well plate. All pipetting actions were implemented in an experimental protocol for automatic liquid handler performance. In step 1, three times 310 μL of each approach was sampled in row A of the plate. The sampling was set on day eleven at midnight and the sedimentation time of each of the four approaches was estimated. The cells were assumed to be in the death phase on that day. Thus, the correction factor and values determined for the death phase were used. Furthermore, the smallest cell diameters detected on day ten were used for the calculations because the sedimentation time of the slowest settling cells was considered. Also, the densities were determined (DMA™ 38 Density Meter, Anton Paar). In step 2, the supernatants were pipetted in row B and 185 μL from there into row D. From a 24-deep-well plate located on the deck next to the 96-well plate, 185 μL of the purification buffers were added to the corresponding wells in step 3. The purification of IgG was then performed in step 4 with an equilibration step and the recommended numbers of cycles (four capture cycles, two times two wash cycles and five elution cycles). In the last step, the liquid handler added 45 μL of neutralisation buffer to the eluates in row G.

### PAIA-Assays for Quantification and Glycan Profiling

The twelve purified samples in row G (see FIG. 3.5) were then analysed for glycosylation structures using high throughput assays from PAIA Biotech. Previous studies revealed that the signal of these assays is higher and clearer for purified antibodies compared to analytes in the supernatant (internal data, not shown).

The first assay was a quantification assay that determined the titers of the analytes. Based on the results, a glycan profiling assay was done with purified IgG samples of 200 μg/mL. The principles of both assays were based on the high affinity of functionalised Protein A beads to IgG and the interaction of IgG with certain fluorescence markers. The dried Protein A beads covered the wells of the provided 384-well plate. Furthermore, the wells had protrusions on the bottom which allowed the separation of marker-IgG-bead complexes from unbound fluorescence markers. Because the fluorescence signal of free markers was measured from the bottom through the clear protrusion, no washing steps were needed in the assays. From the signal of the unbound markers, one could conclude the IgG titers in the quantification assay and the glycan profiles (glycan assay). The read-out was done in a fluorescence microscope (Nyone®, Synentec) with an excitation wavelength of about 640 nm and an emission wavelength of 665 nm. The assays were supplied with the fluorescence marker solutions and other optimised PAIA-mixes. In both assays, all the pipetting actions except dilution steps were executed by the ambr® 15 liquid handler.

### Quantification Assay

For the quantification assay, the purified samples were diluted 1:20 with phosphate-buffered saline (PBS, GE Healthcare) in 1.5 mL tubes (Semadeni). Then, 20 μL of these diluted protein solutions and of a calibration standard were added into wells together with 50 μL of ready-to-use PAIA-mix. The calibration standard was in the same matrix as the pure samples and ranged from 0 μg/mL to 200 μg/mL. Triple determinations of each sample were considered. The data analysis after the read-out was performed with an evaluation tool from PAIA. After generating a calibration curve from the fluorescence signals of the calibration standard, it calculated the corresponding sample concentrations.

### Glycan Assay

Lectins are specific proteins that can bind certain carbohydrate structures. The method of the glycan assay was based on the specific affinity of several fluorescence-marked lectins to different sugar structures at the Fc-site of the IgG and the high affinity of Protein A beads to 3 IgG. Firstly, considering previous studies, the IgG concentration was diluted to 200 μg/mL with PBS. Internal, not presented data showed that this was the optimal condition for the assay. Then, the immunglobulins had to be denatured to expose the sugar structures at the Fc-sites to the lectins. For this, 300 μL of the dilutions were denatured with 300 μL PAIA denaturation mix for 5 minutes at 75° C. (Thermomixer®, Eppendorf). Subsequently, 20 μL of this solution was pipetted into the wells of the 384-well plate. For glycan profiling, 50 μL of lectins were also pipetted into the wells column-wise. The specificities of the used eight lectins are summarised below in table 3-7. Also, negative controls with only dilution buffer and PAIA denaturation mix were considered. All samples and negative controls were pipetted as triple determinations. The filled plate was then shaken at room temperature for 45 minutes at 14000 rpm and after that for 10 minutes at 1000 rpm to ensure mixing of the components. Afterwards, the plate was allowed to stand without agitation where the Protein A-IgG-lectin complexes settled down.

### Overview of the used Lectins and their Specificities. Some Specificities were Tested Twice, but With Different Lectins

| lectin number | specificty |
|---|---|
| 1 | terminal β-galactose |
| 2 | core fucose (1) |
| 3 | core fucose (2) |
| 4 | core mannose |
| 5 | high mannose |
| 6 | terminal α-mannose (1) |
| 7 | terminal α-mannose (2) |
| 8 | G0F^{a} glycan |

Resulting from the mentioned read-out of unbound markers, the measured fluorescence signal of the negative controls was the highest. The percentage amount of the lectins that had bound to the analytes was calculated by the following equation:

$LB = ( 1 - \frac{{I}_{S}}{{I}_{N}} ) 100 \%$

| | | |
|---|---|---|
| LB | lectin binding | [%] |
| I_{S} | fluorescence intensity of sample | [AU] |
| I_{N} | fluorescence intensity of negative control | [AU] |

### High-Performance Liquid Chromatography

IgG was quantified via high-performance liquid chromatography (Dionex™ UltiMate™ 3000 HPLC System, Thermo Scientific). An SEC column was used (Yarra™ 3 μm SEC 3000, Phenomenex). The mobile phase consisted of 100 mM Na2SO4 (Sigma-Aldrich), 50 mM NaH2PO4 (Sigma-Aldrich) and 50 mM Na2HPO4 (Sigma-Aldrich) in arium water (arium® pro ultrapure water system, Sartorius). For preparation, the samples were diluted 1:2 or 1:5 (depending on the available sample volume and titer) with the solution of the mobile phase.

Also, an IgG standard ranging from 0.025 g/L to 2.0 g/L was considered. All samples were then filtered into 1.5 mL autosampler cups (Minisart® RC 4 0.2 μm, Sartorius). In the chromatography method, a flow of 1 mL/min, a column temperature of 25° C. and a maximum pressure of 180 bar was set. UV detection was done at 220, 260 and 280 nm. In this work, only the results of 220 nm were considered though. The absorbance was plotted against the retention time by the software Chromeleon™ 7. The higher the retention time of a molecule in SEC, the smaller it is. The relevant peak of the IgG-monomer appeared at a retention time of around 8.0 minutes. Polymers of IgG were not considered in this work. The concentration of the analyte resulted from the peak area value inserted into the determined calibration curve.

### Feedback Experiment

A feedback experiment was conducted to demonstrate the feasibility of the claimed method. In said experiment, parallel CHO-bioreactor cultivations producing a monoclonal antibody were started. The process and results are shown in FIGS. 7-9.

FIG. 7A shows that at day 4 samples were taken (71) from 3 parallel cultivations and analysed for glycan-profiles (72). Values are in relative to the total amount of measured glycans. The different glycan profile abbreviations are explained in FIG. 7B. As can be seen, in all three cultivations, fucosylated species (FA2, FA2G1, FA2G2) were highly abundant at day 4. A decision was taken to reduce the fucosylated species, for example in order to increase ADCC potency (see e.g. Peipp et al 2008).

For this reason, the feeding strategy was adapted to contain an inhibitor of fucosyltransferase (2F-Peracetly-Fucose) in the feed (73). The feed was administered once per day throughout the experiment. Process samples were taken at the end of cultivation (day 12) and analyzed to check for the effect of the altered feeding strategy and compared to data from standard cultivations (74). The following table shows the percentage distribution of analyzed glycans for the measured antibodies as a result of the modified feeding strategy (average of all 3 cultivations).

| | Day 12 | Day 4 |
|---|---|---|
| It FA2: | 7,3 | 40,5 |
| FA2G1: | 2,6 | 48,1 |
| FA2G2: | 0,26 | 10,7 |

FIGS. 8 and 9 shows the respective data in more details.

### REFERENCES

Zeiser A, Bédard C, Voyer R, Jardin B, Tom R, Kamen A A. Biotechnol Bioeng. 1999 April 5; 63 (1):122-6.

Harris C. M., Todd R. W., Bungard S. H., Lovitt R. W., Morris J. G., Kell D. B. Enzyme Microb. Technol. 1987; 9:181-186. doi: 10.1016/0141-0229 (87)90075-5.

Ohadi, K., Legge, R. L. and Budman, H. M. (2015). Biotechnol. Bioeng., 112: 197-208.

Ohadi, K., Aghamohseni, H., Legge, R. L. and Budman, H. M. (2014). Biotechnol. Bioeng., 111: 1577-1586

Gustaysson R (2018), Linköping Studies in Science and Technology, Dissertation No. 1954

Abu-Absi R et al., Biotechnology and Bioengineering, Vol. 108, No. 5, May 2011

Kroll P et al, Biotechnol Lett. 2017 November; 39 (11):1667-1673

Peuker T et al., Engineering in Life Sciences 4 (2):138-143 April 2004

Luttmann Ret al., Biotechnol J. 2012 August; 7 (8):1040-8.

Konakovsky V et al., Bioengineering (Basel). 2016 March; 3 (1): 5.

McGillicuddy N et al. (2017) Biotechnology Letters 40 (1)

Zhang L et al (1), MAbs. 2016 April; 8 (3): 524-535

Zhang L et al (2), MAbs. 2016 February-March; 8 (2): 205-215

Yang X et al., MAbs. 2016 May-June; 8 (4): 706-717

Strober W, Curr Protoc Immunol 2001 May, Appendix 3

Luo J et al., Biotechnol Bioeng. 2012 September; 109 (9):2306-15

Kimple M E et al, Curr Protoc Protein Sci. 2013; 73: Unit-9.9

Shukla A A et al (2007). Journal of Chromatography B. Polyclonal and Monoclonal Antibody Production, Purification, Process and Product Analytics. 848 (1): 28-39.

Nascimento K S et al, J Mol Recognit. 2012 November; 25 (11):527-41

Coskun O, North Clin Istanb. 2016; 3 (2): 156-160

Prasad K N and Sinha P K. In Vitro. 1976 February; 12 (2):125-32.

Yagi Y et al, J Exp Clin Cancer Res. 2010 Nov. 17; 29:149

Fusi E et al, Italian Journal of Animal Science, Volume 7, 2008—Issue 2

Winckler S et al, Bioprocess Biosyst Eng. 2014 May; 37 (5):901-12

FDA (“Guidance for Industry, November 2009”), https://www.fda.gov/downloads/drugs/guidances/ucm073507.pdf

Peipp Metal., Blood. 2008 Sep. 15; 112 (6):2390-9. doi: 10.1182/blood-2008-03-144600. Epub 2008 Jun. 19.

1. A method for a cell cultivation system, wherein the cultivation system comprises two or more cultivation vessels for the production of at least one biologic agent and/or cell, wherein the cultivation vessels comprise cells in a suitable cultivation medium, the the method comprising the steps of
a) taking two or more liquid samples from two or more or cultivation vessels,
b) optionally, purifying the two or more liquid samples,
c) analyzing at least one of the two or more liquid samples to acquire data relating to at least
one system parameter indicative for at least one of nutrient status and/or medium quality of the cultivation medium, or cell density, or cell viability and/or
one product parameter indicative for biologic agent quality and/or cell quality, and
d) in response to the outcome of step c), adjusting, preferably in real-time, at least one process parameter and/or at least one feeding input in at least one cultivation vessel of the present cultivation system, or of a subsequent cultivation system.

2. The method according to claim 1, wherein the cell cultivation system is a bioreactor system suitable for culturing cells, wherein the bioreactor system comprises two or more bioreactors.

3. The method of claim 1, further comprising transferring at least one of the two or more liquid samples from the cell cultivation system to an array of reaction vessels, wherein the array of reaction vessels comprises at least one of a microtitre plate with two or more wells, a series of sample cups or sample vials, or an array of microreaction tubes.

4. The method of claim 1, wherein the biologic agent is selected from the group consisting of a biomolecule, a cell-based product and a vaccine.

5. The method of claim 1, wherein the process parameter that is adjusted in step d) is at least one of the process parameters selected from the group consisting of: a cultivation temperature set point, stirring speed, stirrer blade tip speed, pH set-point, DO set-point, viable cell density set-point, conductivity, osmolality, specific power input, and bubble size.

6. The method of claim 1, wherein the at least one feeding input that is adjusted in step d) is at least one feeding input selected from the group consisting of: O₂ gassing rate, compressed air gassing rate, CO₂ gassing rate, N₂ gassing rate, input of feed solution, composition of feed solution, addition or omission of individual ingredients in feed solution, addition of protective agents, input of buffer, addition of components related to increased productivity or prevention of cell death, addition of proliferation stimulators, differentiation factors, and addition of specific inhibitors.

7. The method of claim 1, wherein at least one further system parameter is measured on-line in at least one of the cultivation vessels.

8. The method of claim 7, wherein said further system parameter measured on-line is at least one system parameter selected from the group consisting of dissolved oxygen, pH, trypan blue staining, pCO₂, optical density, osmolality, osmolarity, cell permittivity, radio-frequency (RF) impedance, cultivation time, exhaust gas composition, O₂ consumption, temperature, stirrer speed, and metabolite levels.

9. The method of claim 1, wherein the method further comprises, after step c) and before step d), the determination of a soft sensor parameter based on at least one system and/or product parameter, wherein the soft sensor is used as a basis for the adaptation in step d).

10. The the method of claim 1, wherein the two or more liquid samples are taken with at least one of a robotic liquid handler and/or a multi-valve sample system.

11. The method of claim 10, wherein taking of two or more liquid samples is triggered by a given system parameter.

12. The method of claim 10, wherein taking of two or more liquid samples is triggered by a timer.

13. The method of claim 1, wherein at least one of the two or more liquid samples comprises supernatant from one of the cultivation vessels.

14. The method of claim 1, wherein the system parameter indicative for cell density is at least one system parameter selected from the group consisting of: optical density, cell permittivity, radio-frequency (RF) impedance, transmission spectrum, light scattering, and trypan blue staining.

15. The method of claim 1, wherein the system parameter indicative for cell viability is at least one selected from the group consisting of: trypan blue exclusion, cell permittivity, radio-frequency (RF) impedance, and transmission spectrum.

16. The method of claim 1, wherein the system parameter indicative for nutrient status and/or medium quality of the cultivation medium is at least one system parameter selected from the group consisting of glucose concentration, concentration of at least one trace element, vitamin, organic acid or amino acid, osmolality, osmolarity, lactate concentration, acetate concentration, ammonia concentration, and pH value.

17. The method of claim 4, wherein the biomolecule is an antibody or fragment or derivative thereof, an antibody mimetic, a growth factor, a hormone or a cytokine.

18. (canceled)

19. (canceled)

20. The method of claim 1, wherein the product parameter indicative for quality of the cell-based product is at least one selected from the group consisting of presence of one or more given surface markers and cell viability.

21. (canceled)

22. (canceled)

23. The method of claim 1, wherein the purification step comprises at least one step selected from the group consisting of protein A based purification, protein G based purification, affinity tag based purification, lectin based purification, ion chromatography, affinity chromatography, and size exclusion chromatography.

24.-28. (canceled)

29. A method of manufacturing a biologic agent, the method comprising the method of claim 1.

30. (canceled)
